(19) United States
(12) Patent Application Publication
(10) Pub. No.: US 2010/0221240 A1
(43) Pub. Date: Sep. 2, 2010

(54) Chemically Modified Peptide Analogs

(75) Inventors: Afroditi Kapurniotu, Aachen (DE); Jürgen Bernhagen, Aachen (DE)

Correspondence Address: HARNESS, DICKEY & PIERCE, P.L.C., P.O. BOX 828, BLOOMFIELD HILLS, MI, 48303, US

(73) Assignee: FRAUNHOFER-GESELLSCHAFT ZUR FORDERUNG DER ANGEWANDTEN FORSCHUNG E.V., Munchen (DE)

(21) Appl. No.: 11/666,043

(22) PCT Filed: Oct. 18, 2005
(86) PCT No.: PCT/EP2005/011211

§ 371 Date: Apr. 20, 2007

(30) Foreign Application Priority Data
Oct. 20, 2004 (DE) 10 2004 051 014.8

(51) Int. Cl. A61K 38/17; A61K 38/28; A61K 39/395; A61P 3/10; C07K 14/575

(52) U.S. Cl. 424/130.1; 514/12; 514/4; 530/324

(57) ABSTRACT

The present invention relates to improved agents and methods for treating diabetes and Alzheimer's disease by use of IAPP peptide derivatives.

The present invention relates to peptide analogs of islet amyloid polypeptide (IAPP), methods for detecting IAPP or its aggregates, pharmaceutical preparations for the prevention and treatment of protein aggregation disorders, in particular diabetes and Alzheimer's disease, and diagnostic compositions for the detection of protein aggregation disorders, in particular diabetes and Alzheimer's disease, and uses of said peptide analogs for the diagnosis and treatment of medical conditions or for basic research.

Diabetes is a medical condition for which there are currently no satisfactory therapeutic approaches. A distinction is generally made between Type I and Type II diabetes. 90% of diabetics are affected by Type II diabetes, or age-related diabetes. There are currently more than 150 million Type II diabetics worldwide. Diabetes is caused by an insufficiency of insulin or by the malfunction of insulin-dependent biological processes. Since on the one hand the key role of insulin in carbohydrate metabolism cannot be replaced by other molecules, and on the other hand the administration of insulin alone cannot eliminate the pathological effects of the disease in the case of Type 1 diabetes, or the disease itself in the case of Type II diabetes, new therapeutic approaches are necessary. Such approaches should support the secretion, absorption, and development of the biological function of the insulin molecule, and should also provide protection from undesired side effects of insulin, such as weight gain or hypoglycemia. Novel molecules which in particular together with insulin assist in the regulation of carbohydrate metabolism are therefore of great interest in the biomedical field.

One of these novel molecules is islet amyloid polypeptide (IAPP or amylin). IAPP is a peptide hormone, consisting of 37 amino acids, which is synthesized in the β-cells of the pancreas and which, together with insulin and glucagon, is involved in the regulation of sugar metabolism. IAPP is an antagonist of insulin. So-called amyloid plaques are found in the pancreas of more than 95% of Type II diabetics. Amyloid plaques are deposits composed of insoluble aggregates of the polypeptide IAPP. Similarly as for Alzheimer's disease, Parkinson's disease, prion diseases, or Huntington's disease, diabetes may also be regarded as a protein aggregation disorder (Ross and Poirier, Nature Medicine, July 2004, Vol. 10, Suppl., pages 10-17). The bioactivity of IAPP is mediated by its soluble monomeric form via cAMP-coupled receptors. However, IAPP amyloid plaques, soluble oligomers, and multimeric forms of the IAPP molecule are cytotoxic. It is assumed that the damage to the β-cells caused by IAPP amyloid formation makes a significant contribution to the cascade of pathogenesis of Type II diabetes. The development of therapeutically effective inhibitors of the IAPP amyloid formation process, therefore, is of great interest in the biomedical field. Furthermore, in its biological function as an insulin antagonist and as a regulator of postprandial hypoglycemia associated with the secretion or administration of insulin, the IAPP molecule per se is an important polypeptide hormone which could have potential therapeutic applications in the treatment of Type I (e.g., in combination with insulin) and Type II diabetes. However, the medical use of IAPP is greatly limited on account of its poor solubility and pronounced aggregation tendency.

The soluble IAPP analog pramlintide is known. Pramlintide (Symlin®) is synthesized by replacing three amino acids at positions 25, 28, and 29 in the sequence of the human IAPP molecule by proline. These proline substituents occur in the rat IAPP sequence, which has no aggregation tendency. Structurally, the reduced aggregation tendency of proline-containing sequences may be explained by the fact that proline radicals are not able to assume β-fold conformations (prolines are β-fold “breaking” radicals). In fact, the IAPP analog pramlintide has a greatly reduced tendency toward aggregation, and therefore has a better solubility profile than human IAPP. Clinical studies still in progress indicate that this analog together with insulin could find application in the treatment of diabetes. However, the tendency of pramlintide to aggregate at pH values above 5 prevents formulation and administration together with insulin. The analog is therefore administered separately from insulin by s.c. injection, which makes its use much more difficult (Nyholm B. et al., Expert Opinion, Investig. Drug (2001) 10(9) 1641-1652).

By early diagnosis of diabetes together with early onset of treatment it is possible to greatly delay and thus positively influence the progression of the disease. The role of IAPP in Type II diabetes, however, is not fully understood. Since the aggregation of IAPP is dependent on its concentration, there could definitely be a relationship between the concentration of soluble IAPP, i.e., the soluble IAPP aggregates in the blood, and the occurrence of the disease. The conventional IAPP RIAs can only determine the IAPP molecules recognized by the IAPP antibody. However, it is known that protein regions (antigen regions), which are responsible for the antigen-antibody interaction, can no longer be easily recognized by the protein association. It is therefore important to develop a chemical laboratory method for measuring IAPP which is specifically able to recognize various association forms of the IAPP molecule. For this purpose it appears necessary to develop a conformation-specific antibody, i.e., an antibody which has been produced for acting against a monomeric or oligomeric IAPP molecule. The conventional IAPP antibody is not conformation-specific.

The object of the present invention, therefore, is to provide agents and methods by which the prevention, treatment, and diagnosis of protein aggregation disorders, in particular diabetes and/or Alzheimer's disease, may be improved.

This object is achieved by the invention by the preparation of peptide analogs of islet amyloid polypeptide (IAPP) which are able to bind to natural IAPP receptors, whereby a) the peptide analog contains a maximum of 38 amino acids, preferably a maximum of 37 amino acids, of which 37 amino acids have the amino acid sequence of natural IAPP in its natural sequence, b) the peptide analog contains at least amino acids 19 through 37 of natural IAPP in its natural sequence, c) at least one of the amide bonds of the peptide analog is N-methylated, and optionally, d) in the amino acid sequence of the natural IAPP, Lys (lysine) at position 1 may be replaced by Orn (ornithine), and/or Cys (cysteine) at position 2 and 7 may be replaced by Dap (2,3-diaminopropionic acid) at position 2 and by Asp (asparaginic acid) at position 7, or by Asp at position 2 and Dap at position 7, and e) the N-methylated peptide analogs with SEQ ID No. 1 through 5 having the wild type amino acid sequence of human IAPP are extracted. The object of the present invention is achieved in particular by the preparation of a peptide analog of islet amyloid polypeptide (IAPP) as a diagnostic or therapeutic agent which is able to bind to natural IAPP receptors, whereby a) the peptide analog contains a maximum of 38 amino acids, preferably a maximum of 37 amino acids, of which 37 amino acids have the amino acid sequence of natural IAPP in its natural sequence, b) the peptide analog has at least amino acids 19 through 37 of natural IAPP in its natural sequence, c) at least one of the amide bonds of the peptide analog is N-methylated, and optionally, d) in the amino acid sequence of the natural IAPP, Lys (lysine) at position 1 may be replaced by Orn (ornithine), and/or Cys (cysteine) at position 2 and 7 may be replaced by Dap at position 2 and by Asp at position 7, or by Asp at position 2 and Dap at position 7.

According to the invention, these peptide analogs, i.e., the modified N-methylated IAPP derivatives, are preferably prepared in isolated, preferably in partially or completely purified, form.

The above-referenced peptide analogs are therefore characterized by the absolutely necessary features a), b), and c), and may optionally contain feature d); i.e., in this optional embodiment of the present invention the peptide analogs are characterized by an amino acid sequence which deviates from the natural IAPP sequence, in particular human IAPP, by the fact that lysine, which is contained at position 1 in the natural human IAPP sequence, is replaced by the amino acid ornithine, and/or the two cysteines at position 2 and 7 of natural, in particular human, IAPP are replaced by Dap (2,3-diaminopropionic acid) at position 2 and by Asp at position 7, or by Asp at position 2 and Dap at position 7. A peptide analog according to the invention which is provided according to feature d), i.e., in a deviation from the natural IAPP sequence, in particular the human IAPP sequence, contains ornithine at position 1 (instead of lysine) and/or contains Asp or Dap at position 2 and contains Dap or Asp at position 7 (in each case, instead of cysteine) is referred to below as a derivative of the peptide analog according to the invention.

The peptide analogs according to the invention which contain Cys at position 2 and 7 are preferably oxidized; i.e., the disulfide bridge between the thiol radicals of Cys 2 and Cys 7 is closed.

Likewise, the peptide analogs according to the invention which contain Dap and Asp, or Asp and Dap, at position 2 and 7 are preferably bridged; i.e., the side chains of Asp and Dap are covalently bonded to one another via a lactam bridge.

In one preferred embodiment the C-terminus is present as amide In all peptide analogs of the present invention.

In conjunction with the present invention, the term “natural IAPP” is understood to mean IAPP which has the wild type IAPP amino acid sequence, i.e., the amino acid sequence which naturally occurs in vivo in the affected organism, i.e., preferably in humans. Various natural IAPP sequences, in particular for humans, are published in Kapurniotu (Biopolymers (Peptide Science) 60 (2001) 438-459). SEQ ID No. 18 from the present teaching exhibits the wild type IAPP sequence for humans. Within the scope of the present invention, therefore, position data for amino acids or amide bonds, unless stated otherwise, always refer to the amino acid sequence of natural human IAPP, illustrated in diagram 1 by Kapurniotu (above) or in SEQ ID No. 18. Likewise, the term “natural IAPP receptors” is understood to mean naturally occurring wild type IAPP receptors, in particular human wild type receptors.

In one embodiment, the peptide analogs according to the invention have exactly the same primary structure as natural human IAPP, and in another embodiment the peptide analogs have essentially the same primary structure as in particular human IAPP, but on account of the N-methyl groups selectively inserted according to the invention at certain amide bonds, have a different conformation than IAPP, and therefore have modified biological, in particular biochemical and biophysical, properties compared to native human IAPP. The peptide analogs according to the invention are characterized in particular by the fact that they are able to interact with native IAPP, in particular native human IAPP, and to reduce or inhibit the aggregation thereof in amyloid fibriles, the subsequent amyloid plaque formation, and the associated cytotoxicity. At the same time, said peptide analogs have the advantageous property of being able to bind specifically to natural, i.e., wild type, receptors, i.e., preferably human IAPP receptors. The peptide analogs according to the invention have no, or a greatly reduced, capability for fibrile formation. At a physiological pH of approximately 7, they show a particularly greatly reduced tendency toward fibrile formation and thus, in contrast to native IAPP or pramlintide, may be formulated and administered together with insulin in a particularly advantageous manner. The peptide analogs according to the invention, in particular at a physiological pH of approximately 7 to 7.4, are least a hundred times more soluble than IAPP, which allows them to be formulated with insulin as described above. As a result of their high solubility in comparison to IAPP, their biological activity is not reduced during storage as a solution (1 mg/mL), which is the case with IAPP. In addition, as an additive to IAPP-containing solutions they can assist in keeping the IAPP in soluble, and therefore biologically active, form. Compared to IAPP, the peptide analogs according to the invention are more stable against degradation by proteases, which could also allow administration of the peptide analogs of the present invention in tablet form. Furthermore, the peptide analogs according to the invention interact with β-amyloid peptide, which plays a role in the development of Alzheimer's disease. The peptide analogs according to the invention reduce in particular the cytotoxicity of β-amyloid peptide, and are therefore suited for the diagnosis, treatment, and prevention of Alzheimer's disease.

The peptide analogs according to the invention are particularly suited for the prevention, treatment, and diagnosis of protein aggregation disorders, in particular diabetes, particularly preferably Type I diabetes and/or Type II diabetes (diabetes mellitus). In one preferred embodiment, the peptide analogs according to the invention are optionally used together with insulin and/or IAPP and/or pramlintide for the prevention and treatment of diabetes, in particular Type I and/or Type II diabetes. Furthermore, the conformation-stabilized peptide analogs prepared according to the invention may be used as antigens, e.g., alone or in a mixture with IAPP monomers or IAPP oligomers of defined size, in order to prepare and use conformation-specific antibodies for, e.g., ELISA/RIA-based detection of IAPP in bodily fluids, thereby providing improved diagnostic and analytical methods.

According to the invention, the peptide analogs according to the invention may be used, e.g., for ELISA, RIA, etc., together with a marker, e.g., a spin label or fluorescence or luminescence marker, in particular in an N-terminal-labeled, particularly preferably in an N-terminal biotinylated, form, or in N-terminal fluoroescein-labeled form, or provided with other fluorescent markers.

In one preferred embodiment, the invention provides that the peptide analog according to the invention contains the complete amino acid sequence 1 through 37 of natural IAPP, in particular of natural human IAPP, or contains amino acids 1 through 37 of the above-referenced derivative thereof or contains these 37 amino acids alone, i.e., consists of same. In one particularly preferred embodiment, the invention thus provides a peptide analog as referenced above which comprises the 37 amino acids of natural, in particular human, IAPP, or the amino acids of the above-referenced derivative, which are configured in the sequence of natural human IAPP, whereby at least one of the amide bonds of the α-amino groups of the amino acids in this peptide analog is N-methylated.

In one particularly preferred embodiment, the invention provides the teaching that the peptide analog of IAPP according to the invention, which comprises the complete amino acid sequence of natural, in particular human, IAPP, namely, the amino acid sequence 1 through 37 in its full length, or has the amino acid sequence of the above-referenced derivative thereof or consists of same, acts as a receptor agonist of the natural IAPP receptor. In this embodiment the peptide analog activates the IAPP receptor. According to this teaching, these peptide analogs according to the invention are particularly suitable for regulating sugar metabolism and other biological activities of the native IAPP molecule. At the same time, these peptide analogs act as inhibitors for the formation of amyloid fibriles and/or amyloid plaques, i.e., as inhibitors for the formation of insoluble cell-damaging IAPP aggregates, and thus simultaneously reduce or inhibit the cytotoxicity caused thereby.

In one further preferred embodiment, the invention provides that in comparison to the wild type sequence the peptide analog according to the invention is shortended, in particular at its N-terminus, and contains at least amino acids 19 through 37, preferably 8 through 37, of natural IAPP, or the amino acids of the above-referenced derivative thereof, or consists solely of these amino acids in their natural sequence. Also in this embodiment, the shortened peptide analog contains at least amino acids 19 through 37, preferably 8 through 37, and at a maximum, amino acids 2 through 37 of natural, in particular human, wild type IAPP, or contains the amino acids of the above-referenced derivative thereof or consists of same, and at least one of the amide bonds of the peptide analog is N-methylated.

The invention also provides the teaching that the shortened peptide analogs according to the invention which do not have the complete amino acid sequence of natural, in particular human, IAPP, or do not have the complete amino acid sequence of the above-referenced derivative, i.e., the peptide analogs which contain or consist of the amino acids from positions 2 through 37 to positions 19 through 37, or the amino acid sequence of the above-referenced derivatives thereof, likewise bind to a wild type natural IAPP receptor, but at that location act as receptor antagonists and are therefore likewise suited to function as regulators of sugar metabolism. In this embodiment the peptide analog inhibits the IAPP receptor. These particularly preferred peptide analogs also simultaneously act as inhibitors for the formation of amyloid fibriles and/or amyloid plaques, and therefore reduce or inhibit the associated cytotoxicity.

Thus, the invention also encompasses embodiments, i.e., peptide analogs of wild type IAPP, which, e.g., contain amino acids 7 through 37, 6 through 37, 5 through 37, 4 through 37, 3 through 37, and 2 through 37 of natural, in particular human, IAPP, or the amino acids of the above-referenced derivative thereof, or consist solely of same, whereby in each of these embodiments at least one of the amide bonds of the peptide analog is N-methylated.

In one preferred embodiment, the peptide analogs according to the invention have one, two, three, or four amide bonds which are N-methylated, i.e., in which a hydrogen atom of the α-amino group of an amino acid of the peptide is replaced by a methyl group. In one preferred embodiment of the present invention, the amide bonds of the peptide analogs according to the invention which are associated with positions 22 and/or 23 and/or 24 and/or 25 and/or 26 and/or 27 and/or 28 and/or 29 are N-methylated. In one particularly preferred embodiment, the N-methylated amide bonds are located in the amino acid sequence in a region of the peptide analog which contains 4 to 8 amino acids in a continuous series. In a further preferred embodiment, the N-methyl groups may be present in a region of the peptide analog which contains 4 to 8 amino acids at every second amide bond of the peptide analog, in particular at the above-referenced amino acid positions.

In one particularly preferred embodiment, the present invention relates to peptide analogs selected from the group comprising SEQ ID No. 1 through 17, with the exception of peptide analogs which have the natural amino acid sequence of human wild type IAPP and at the same time have the N-methylation according to SEQ ID No. 1 through 5. In a further preferred embodiment the present invention relates to peptide analogs for therapeutic or diagnostic purposes, selected from the group comprising SEQ ID No. 1 through 17. For the peptide analogs according to the invention, in particular for SEQ ID No. 1 through 17, the invention thus provides the first application in a method for therapeutic treatment of the human or animal body, and in a diagnostic method which is performed on the human or animal body. The invention therefore also relates to pharmaceutical and diagnostic agents which contain the above-referenced peptide analogs of the invention, in particular the peptide analogs selected from the group comprising SEQ ID No. 1 through 17.

In one particularly preferred embodiment, the present invention relates to peptide analogs selected from the group of peptide analogs according to the invention containing amino acids 1 through 37 of human wild type IAPP or of a derivative thereof, which contain the amide bonds of the α-amino groups of the amino acids at the positions (24 and 26), (25 and 27), (23 and 25), (26 and 27), (25 and 26 and 27), (24 and 25), (25 and 26), (22 and 24), (23 and 24), (24 and 26), (23), (24), (25), (26), (27), (28 and 29), (25 and 28 and 29). These peptide analogs are also referred to below in the above-referenced sequence as IAPP-GI, IAPP-AL, IAPP-FA, IAPID-IL, IAPP-AIL, IAPP-GA, IAPP-AI, IAPP-NG, IAPP-FG, IAPP-GAIL, IAPP-F, IAPP-G, IAPP-A, IAPP-I, IAPP-L, IAPP-SS, and IAPP-ASS, provided that they have the amino acid sequence of human wild type IAPP.

Of course, the invention also relates to the above-referenced peptide analogs of wild type IAPP or its derivatives in a mixture, such as a 1:1 mixture, with natural unmodified IAPP and/or β-amyloid peptide or other known peptide analogs of IAPP, or of β-amyloid peptide (A-β) or other derivatives of IAPP or A-β, in particular also for producing pharmaceutical or diagnostic agents and for research and testing purposes.

In one further preferred embodiment, the invention relates to the peptide analogs according to the invention which are soluble, in particular at least a hundred times more soluble than natural IAPP, in particular human natural IAPP, in particular in water or physiological saline solution, preferably at a physiological pH of approximately 7.0 to 8.0, preferably pH=7.4.

In one further preferred embodiment, the peptide analogs according to the invention are labeled, in particular at their N-terminal α-amino group, in particular with an acetyl group, a radioactive marker, an enzyme marker, a fluorescent marker, a luminescent market, or a spin label, preferably in such a way that said marker is joined to the peptide analog by means of a spacer, which may be an amino acid.

In one further preferred embodiment, the peptide analogs according to the invention are derivatized with at least one functional group selected from the group comprising an acyl group, a functionalized acyl group, an aromatic group, an amino acid, a glycol group, and a lipid group.

In one preferred embodiment, the functional group is joined to the peptide analog by means of a spacer, e.g. an amino acid, provided that the functional group itself is not an amino acid group.

In one further preferred embodiment, the peptide analogs according to the invention are immobilized on a substrate, e.g., a matrix, or on the surface of a well, a microtiter plate, a membrane, etc.

The peptide analogs according to the invention may generally be chemically synthesized and/or modified.

The invention also relates to a method for producing antibodies directed specifically against a) IAPP or b) a peptide analog/IAPP complex or directed specifically against c) a peptide analog of the present invention, whereby at least one peptide analog according to the invention in the form of an antigen, optionally together with monomeric IAPP or an oligomer thereof, is brought into contact with a system which is capable of forming antibodies, e.g., injected into an animal organism, and the antibodies which form are recovered. Of course, the invention also relates to methods for producing antibodies directed specifically against IAPP or an IAPP/peptide analog complex or directed specifically against a peptide analog of the present invention, whereby the antibodies may be monoclonal as well as polyclonal antibodies. The invention therefore also relates to monoclonal or polyclonal antibodies or fragments thereof which may be produced according to a method described above, whereby the antibody or fragment thereof is directed specifically against a peptide analog of the present invention or directed specifically against IAPP or a peptide analog/IAPP complex, i.e., is able to specifically recognize and bind to same. The antibodies may be modified in the usual manner, e.g., labeled. They may also be present in immobilized form, fixed on a substrate or a pellet. The polyclonal or monoclonal antibodies according to the invention may be used, e.g., for analyzing the disease progression in patients treated with peptide analogs according to the invention, e.g., or for isolating and identifying additional therapeutically effective peptides. With regard to their use as immunogens, the peptides according to the invention have proven to be particularly advantageous for the production of antibodies for diagnostic and therapeutic purposes on account of their greatly improved manageability compared to the natively occurring, poorly soluble peptides. In one embodiment, the antibodies thus produced may specifically recognize the peptides according to the invention, and in another embodiment may also recognize the natively occurring wild type IAPP, optionally in aggregated form, so that the antibodies according to the invention may be used, e.g., for diagnosis of Alzheimer's disease or diabetes. The invention further relates to methods for immunizing human or animal organisms, whereby the peptide analogs according to the invention are administered to human or animal organisms and immunization is achieved against IAPP or its derivative.

The invention therefore relates to methods for producing antibodies directed specifically against a peptide analog of the present invention, whereby at least one peptide analog of the present invention, in the form of an antigen, is brought into contact with a system which is capable of forming antibodies, and the antibodies which form are recovered. The invention further relates to a method for producing antibodies directed specifically against IAPP and a peptide analog of the present invention, whereby at least one peptide analog of the present invention is brought into contact with a system which is capable of forming antibodies, and the antibodies which form are recovered. The invention further relates to methods for producing antibodies directed specifically against mixtures of IAPP with a peptide analog, whereby mixtures of monomeric IAPP and a peptide analog of the present invention in the form of an antigen, the IAPP optionally also being usable in the form of an oligomer, are brought into contact with a system which is capable of forming antibodies, and the antibodies which form are recovered. The invention further relates to a method for producing antibodies directed specifically against mixtures of β-amyloid peptide with a peptide analog of the present invention, whereby mixtures or monomeric or oligomeric β-amyloid peptide with at least one peptide analog of the present invention in the form of an antigen are brought into contact with a system which is capable of forming antibodies, and the antibodies which form are recovered.

The invention further relates to specific antibodies or specific fragments thereof, produced by use of the above-referenced methods, which specifically recognize and bind to their antigen, in particular also for the diagnosis, prevention, or treatment of disorders, in particular protein aggregation disorders, in particular diabetes and/or Alzheimer's disease. The invention therefore also relates to the use of the above-referenced antibodies or specific fragments thereof for producing a diagnostic or pharmaceutical agent, in particular for the diagnosis, prevention, or treatment of diabetes and/or Alzheimer's disease.

In one further preferred embodiment, as previously described the present invention relates to a pharmaceutical agent containing at least one peptide analog and/or an antibody of the present invention as active substance, preferably in a prophylactically or therapeutically effective quantity, preferably together with at least one pharmaceutically acceptable carrier.

In one particularly preferred embodiment, this pharmaceutical agent optionally also contains, if necessary, separating agents, lubricants, solvents, dispersants, coatings, antibacterial or antifungicidal agents, preservatives, colorants, emulsifiers, flavorants, or other common formulation adjuvants. Additional substances may be added to the pharmaceutical composition which are used, e.g., for transport in the target organism, e.g. through the blood-brain barrier.

In one further preferred embodiment, the pharmaceutical agent is provided in the form of a depot medication, i.e., which allows slow release of the active substance, i.e., the peptide analog present, and contains, e.g., a slow-release matrix, or whereby the pharmaceutical agent is enclosed in a dragée covering which slowly dissolves in the body of the patient.

In one particularly preferred embodiment, the pharmaceutical agent of the type described above is provided as a combination medication, i.e., also contains insulin and/or IAPP and/or pramlintide in the same formulation or in the same medication pack. The invention therefore also relates to a medication kit containing a) a pharmaceutical formulation containing at least one of the above-referenced peptide analogs and/or an antibody against same and b) a pharmaceutical formulation containing insulin and/or IAPP and/or pramlintide, each optionally provided together with pharmaceutically acceptable carriers and other formulation adjuvants, whereby the peptide analogs and insulin and/or pramlintide and/or IAPP as active substances are present in a prophylactically or therapeutically effective quantity.

In one further preferred embodiment, the invention provides that the pharmaceutical agent is provided in tablet form, as an aerosol, or as a solution, in particular an injection solution.

In one further preferred embodiment, the invention relates to the use of a peptide analog according to the invention or an antibody referenced above for producing a pharmaceutical agent for the prevention or treatment of protein aggregation disorders, in particular diabetes, preferably Type I diabetes or Type II diabetes.

The invention further relates to the use of a peptide analog according to the invention or an antibody referenced above together with insulin and/or IAPP and/or pramlintide for producing a combination medication kit, e.g., a joint formulation or a medication kit for simultaneous or time-released administration using two separate formulations of the peptide analog and/or insulin and/or IAPP and/or pramlintide as active substances for the prevention or treatment of diabetes, e.g., Type 1 or Type 2 diabetes.

In one particularly preferred embodiment, the present invention relates to the use of a peptide analog according to the invention, optionally together with insulin and/or IAPP and/or pramlintide, for producing a pharmaceutical agent for the prevention or treatment of diabetes, e.g., Type 1 or Type 2 diabetes, whereby the peptide analog according to the invention, in particular the peptide analog containing 1 to 37 amino acids of natural IAPP or the amino acid sequence of the derivative thereof, is used as a receptor agonist for the IAPP receptor-controlled treatment of diabetes, in particular for activation of the natural IAPP receptor. The use according to the invention thus provides that within the scope therein the peptide analog of the type described above binds to a natural IAPP receptor in vivo and activates same, thereby allowing the sugar metabolism to be regulated.

In one further preferred embodiment, a use according to the invention is provided in which a peptide analog of the present invention, in particular a shortened, in particular an N-terminally shortened, peptide analog, preferably a peptide analog which contains at least 8 through 37 or at least 19 through 37 and a maximum of 2 through 37 amino acids of natural human IAPP or is an above-referenced derivative thereof, for producing a pharmaceutical agent, optionally together with insulin and/or IAPP and/or pramlintide, is provided for the treatment of diabetes, whereby the peptide analog is used as a receptor antagonist for the IAPP receptor-controlled treatment of diabetes, i.e., binds to and inhibits the natural IAPP receptor in vivo, thereby allowing the sugar metabolism and the other physiological functions of IAPP to be regulated.

In one further preferred embodiment, all peptide analogs according to the invention or antibodies against same are used as inhibitors of IAPP aggregation, in particular as inhibitors of IAPP amyloid plaque formation. The invention also provides that the peptide analogs according to the invention or antibodies against same are used for reducing or inhibiting the cytotoxicity of IAPP, in particular for producing an appropriate pharmaceutical agent. In one further preferred embodiment, the peptide analogs of the present invention or an antibody of the present invention is/are used for producing a pharmaceutical agent for simultaneously a) reducing or inhibiting IAPP aggregation, i.e., formation of amyloid plaques, and b) regulating the sugar metabolism, whether as a receptor agonist or receptor antagonist.

In one further preferred embodiment, a peptide analog according to the invention or an antibody according to the invention is used to produce a pharmaceutical agent, whereby the peptide analog or the antibody is used for simultaneously a) reducing or inhibiting the cytotoxicity of IAPP or the aggregates thereof, and b) regulating the sugar metabolism, whether as a receptor agonist or receptor antagonist.

In one further preferred embodiment, the invention relates to a peptide analog of the above-referenced type and/or an antibody against same which is used to produce a pharmaceutical agent for the prevention or treatment of Alzheimer's disease or other protein aggregation disorders such as Parkinson's disease, Huntington's disease, or prion diseases, and the peptide analog and/or antibody is used in a therapeutically or prophylactically effective quantity, and in particular the peptide analog or antibody against same reduces or inhibits aggregate formation or amyloid plaque formation of β-amyloid peptide, or reduces or inhibits the cytotoxicity thereof.

In one further preferred embodiment, the present invention relates to a peptide analog of the above-referenced type or an antibody against same for producing a pharmaceutical agent for the simultaneous prevention and treatment of a) Alzheimer's disease or other protein aggregation disorders such as prion diseases, Parkinson's disease, or Huntington's disease, and b) diabetes, in particular Type I diabetes or Type II diabetes.

In one further preferred embodiment, the pharmaceutical agent for the prevention and treatment of Alzheimer's disease or other protein aggregation disorders such as Parkinson's disease, prion diseases, or Huntington's disease, in particular for the simultaneous prevention and treatment of a) Alzheimer's disease or other protein aggregation disorders such as prion diseases, Parkinson's disease, or Huntington's disease, and b) diabetes, is provided as a combination medication together with insulin, pramlintide, or IAPP.

A further preferred embodiment of the present invention relates to a peptide analog or an antibody of the present invention for producing a diagnostic agent for the diagnosis of Alzheimer's disease or other protein aggregation disorders such as prion diseases, Parkinson's disease, and/or Huntington's disease.

A further preferred embodiment of the present invention relates to a peptide analog of the present invention or an antibody of the present invention for producing a diagnostic agent for the diagnosis of protein aggregation disorders, in particular diabetes.

The invention further relates to the use of the present peptide analogs for research purposes.

In one preferred embodiment, the present invention relates to a peptide analog of the present invention as a more easily manageable immunogen for the production of antibodies, in particular monoclonal or polyclonal antibodies, for diagnostic, therapeutic, and research purposes.

In one further embodiment the present invention relates to a method for the qualitative and/or quantitative detection of IAPP or the aggregates thereof, whereby a peptide analog of the present invention provided with a detection marker, or an antibody of the present invention provided with a detection marker, in the form of a probe is brought into contact in vivo, i.e., in a human or animal organism, or in vitro with a sample to be examined, and binding of the peptide analog or antibody to IAPP or the oligomers or aggregates thereof which may be present is detected.

There is evidence that soluble oligomers composed of various amyloidogenic polypeptides, i.e., proteins such as β-amyloid peptide, prion protein, polyglutamine (Huntington's disease), α-synuclein (Parkinson's disease), and others have a similar spatial structure (Kayed et al., Science (2003), 300, 486-489). Kayed et al. (loc. cit.) recently showed that an antibody produced for acting against a model of the soluble oligomeric structure of β-amyloid peptide (Alzheimer's disease) is able to recognize soluble, toxic oligomers from various other proteins, such as IAPP, α-synuclein, and prion protein fragments, and neutralize their cytotoxicity in vitro. The analogs according to the invention or the antibodies against same are therefore also suited for various methods for modifying the aggregation or for detecting other amyloidogenic polypeptides such as β-amyloid peptide, prion protein, polyglutamine, or α-synuclein.

The invention further relates to methods for tracking and modifying the aggregation, in particular for the qualitative or quantitative detection, of amyloidogenic peptides, oligomers, or aggregates thereof, in particular IAPP peptides, IAPP oligomers, or IAPP aggregates, or for inhibiting the cytotoxicity of IAPP peptides, oligomers, or aggregates thereof, whereby the peptide analogs according to the invention or antibodies against same are brought into contact in vivo or in vitro with the amyloidogenic peptides, oligomers, or aggregates thereof, and the aggregation behavior of the amyloidogenic peptides, oligomers, or aggregates thereof is modified, and in particular the aggregation may be reduced or inhibited and/or thereby tracked (diagnosis).

The invention further relates to methods for tracking and modifying the aggregation, in particular for the qualitative or quantitative detection, of amyloidogenic peptides, oligomers, or aggregates thereof, in particular β-amyloid peptide, prion protein, α-synuclein, polyglutamine, or oligomers of aggregates thereof, or for inhibiting the cytotoxicity of β-amyloid peptide, prion protein, α-synuclein, or polyglutamine, or oligomers or aggregates thereof, whereby the peptide analogs according to the invention or antibodies against same are brought into contact in vivo or in vitro with the amyloidogenic peptides, oligomers, or aggregates thereof, and the aggregation behavior of the amyloidogenic peptides, oligomers, or aggregates thereof is modified, and in particular the aggregation may be reduced or inhibited and/or thereby tracked (diagnosis).

In one further preferred embodiment the present invention relates to a method for modifying, in particular preventing, the aggregate formation, of IAPP, polyglutamine, α-synuclein, prion protein, or β-amyloid peptide present in a liquid, whereby a peptide analog of the present invention is brought into contact with the liquid and incubated, and the aggregation behavior of the IAPP, prion protein, α-synuclein, polyglutamine, or β-amyloid peptide is modified.

Further embodiments of the present invention result from the subclaims.

### Sequence Protocol:

The SEQ ID numbers show the following:

SEQ ID No. 1 through 17 represents preferred embodiments of the present peptide analogs. Each of the SEQ ID numbers represents a plurality of various peptide analogs of the present invention which for the same N-methylation pattern differ in their primary structure. Each individual SEQ ID number therefore represents a specific N-methylation pattern, which may occur in various primary structures, i.e., for various amino acid sequences, e.g. the natural wild type amino acid sequence of human IAPP or a derivative thereof as defined above, i.e., a derivative according to which the lysine at position 1 and/or the two cysteine radicals at position 2 and 7 [are replaced] by ornithine (as a replacement for lysine) at position 1, and/or asparaginic acid and Dap are used (as a replacement for the two cysteine radicals). Each individual SEQ ID number therefore represents the primary structure of the natural human wild type IAPP having a specific N-methylation pattern, and also represents the above-referenced derivatives having the same N-methylation pattern. The peptide analogs according to the invention which contain the Cys at position 2 and 7 are preferably oxidized; i.e., the disulfide bridge between the thiol radicals of Cys 2 and Cys 7 is closed.

Likewise, the peptide analogs according to the invention which contain Dap and Asp, or Asp and Dap, at position 2 and 7 are preferably bridged; i.e., the side chains of Asp and Dap are covalently bonded to one another via a lactam bridge. In one preferred embodiment of the invention the C-terminus is present as amide for all peptide analogs of the present invention.

An association of the SEQ ID numbers with the abbreviations of the preferred peptide analogs used in the following examples is provided in Example 8.

SEQ ID No. 18 represents the amino acid sequence of natural human IAPP.

The invention is explained in greater detail with reference to the following examples and the accompanying figures, which show the following:

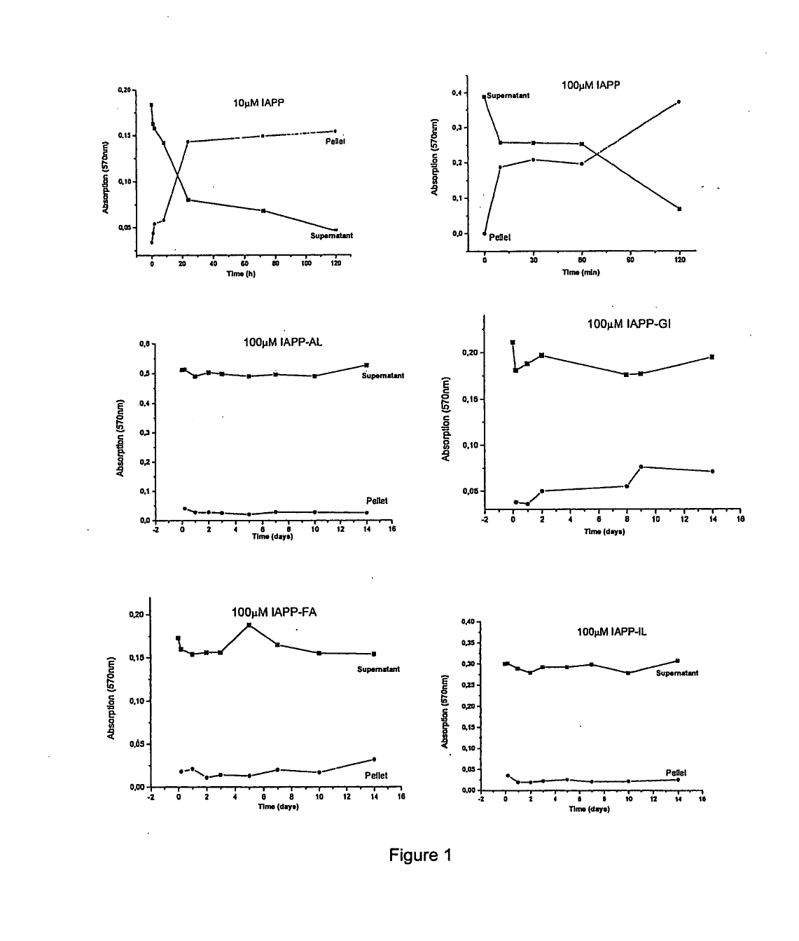

FIG. 1 shows absorptions of sedimentation assays of IAPP (at 10 and 100 μM) and the IAPP-GI, IAPP-AL, IAPP-FA, and IAPP-IL analogs. The absorption at 570 nm corresponds to the quantity of protein (in the pellet or supernatant).

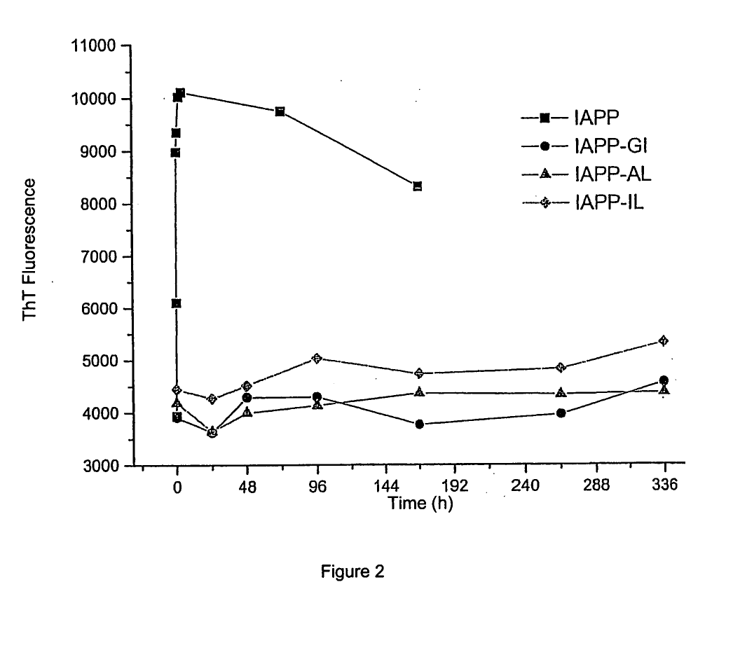

FIG. 2 shows the results of a fibrile formation test: The fibrile formation potential of 62.5 μM IAPP versus 62.5 μM IAPP-GI, IAPP-AL, and IAPP-IL was quantified using the ThT binding assay.

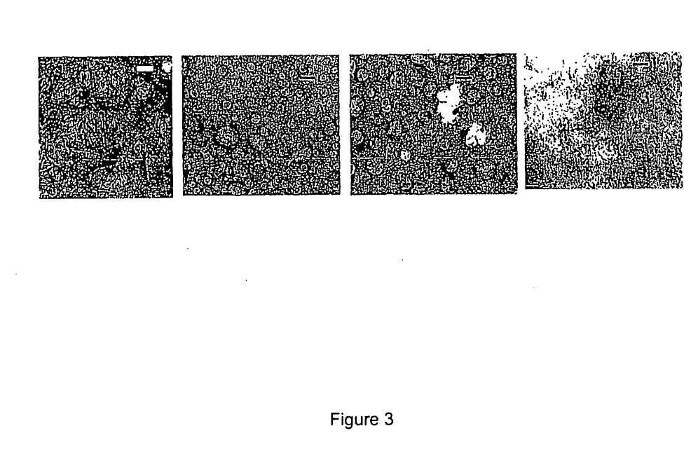

FIG. 3 shows the results of an electron microscopy (EM) test of the amyloidogenic potentials of IAPP and the IAPP-GI, IAPP-AL, and IAPP-FA analogs. The incubations of the peptides (5 μM, 20 h) were examined by an EM-based aggregation test. Aliquots of the following incubations are shown from left to right: IAPP alone, IAPP-GI, IAPP-FA, and IAPP-AL (bar: 100 nm).

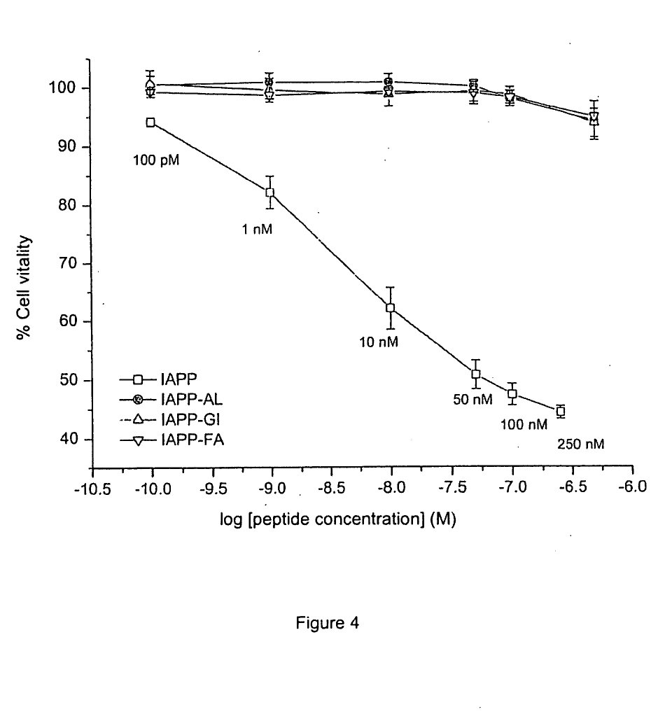

FIG. 4 shows the results of MTT reduction assays for determining the potential cytotoxic effects of the IAPP-GI, IAPP-AL, and IAPP-FA analogs compared to IAPP.

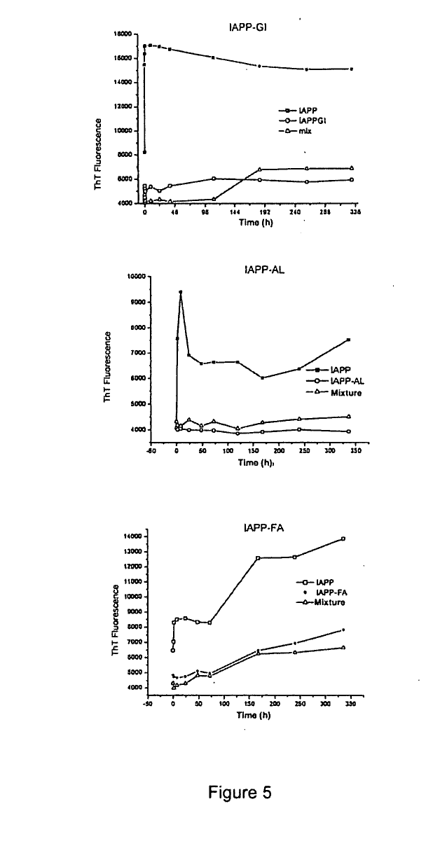

FIG. 5 shows the results of a fibrile binding test: The effect of IAPP-GI, IAPP-AL, and IAPP-FA (1:1 mixtures) on the fibrile formation potential of IAPP (6.25 μM) were quantified by the ThT binding assay.

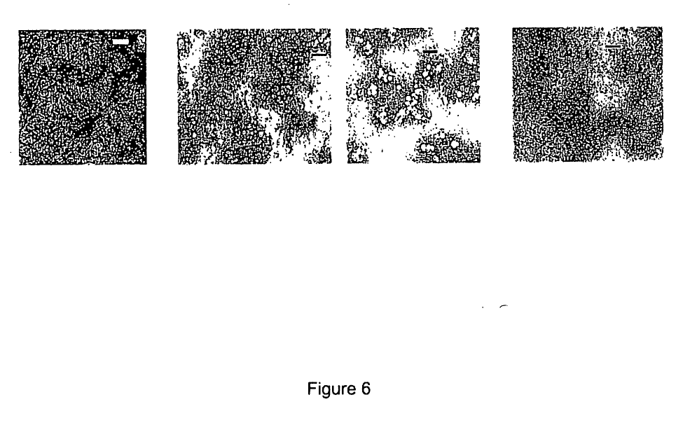

FIG. 6 shows the results of an aggregation test: The effect of IAPP-GI, IAPP-AL, and IAPP-FA (1:1 mixtures) on the fibrile formation potential of IAPP (5 μM) was examined by an EM-based aggregation assay. Aliquots of the following incubations are shown from left to right (20 h): IAPP alone, IAPP mixed with IAPP-GI, IAPP mixed with IAPP-FA, and IAPP mixed with IAPP-AL.

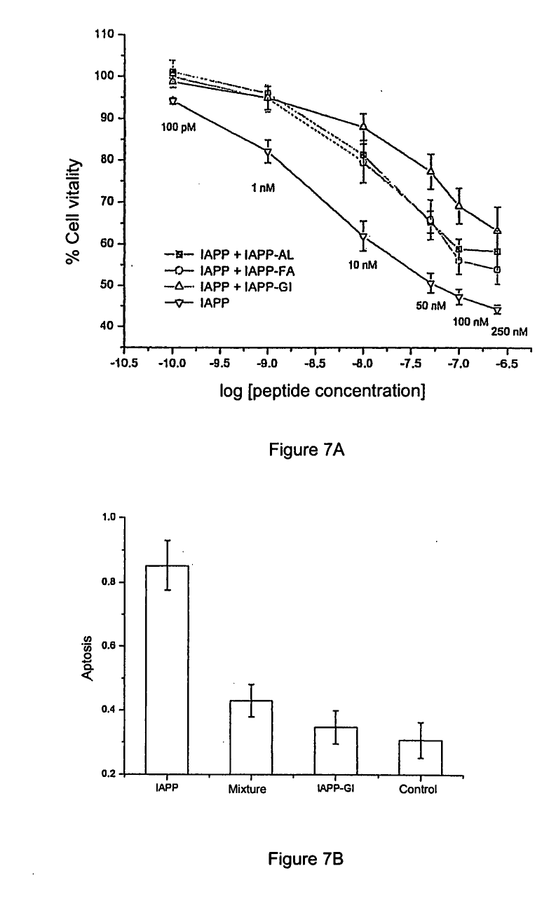

FIG. 7A shows the results of MTT reduction assays for determining the effect of the IAPP-GI, IAPP-AL, and IAPP-FA analogs on the pancreatic cytotoxicity of IAPP (RIN5fm cell line).

FIG. 7B shows the determination of IAPP-induced adoptosis on RIN5fm cells alone and in the presence of IAPP-GI (1:1). IAPP-GI alone was also tested under the same conditions, and no cytotoxicity was found. The corresponding average values (±SEM) from at least two independent assays are shown.

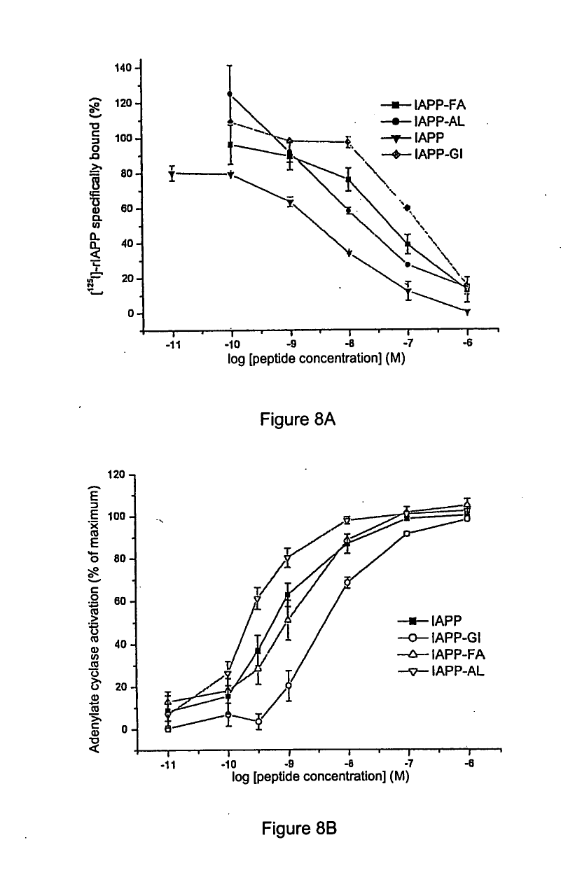

FIG. 8B shows the results of (human) IAPP receptor binding assays using IAPP and the IAPP-GI, IAPP-AL, and IAPP-FA analogs on MCF-7 cells. The specific binding of the radioligand [125I]-rIAPP is plotted versus the ligand concentration.

FIG. 8B shows the results of receptor activation assays using IAPP and the IAPP-GI, IAPP-AL, and IAPP-FA analogs on MCF-7 cells. The adenylate cyclase activation (% of maximum) was determined by quantifying cAMP using the cAMP Biotrak ELISA (Amersham). The maximum AC activation was assumed to be the AC activation achieved by 1 μM IAPP.

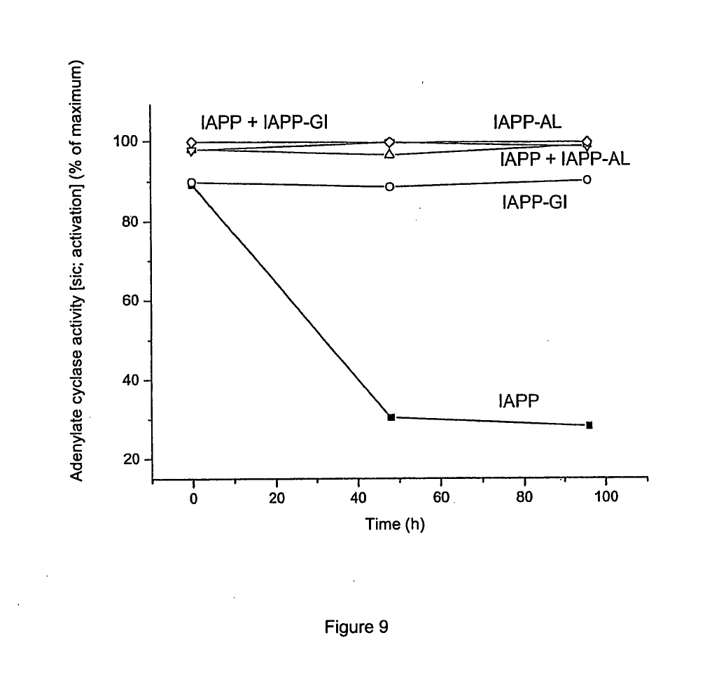

FIG. 9 shows the results of an ELISA: The time dependency of the receptor activation potential on MCF-7 cells in a 250-μM IAPP solution (in 10 mM sodium phosphate, pH 7.4) allowed to stand for 4 days at room temperature (RT) was compared to that for the IAPP-GI and IAPP-LA [sic; IAPP-AL] analogs and mixtures of the analogs with IAPP. The adenylate cyclase activation (% of maximum) was determined by quantifying cAMP using the cAMP Biotrak ELISA (Amersham). The maximum AC activation was assumed to be the AC activation achieved by 1 μM IAPP.

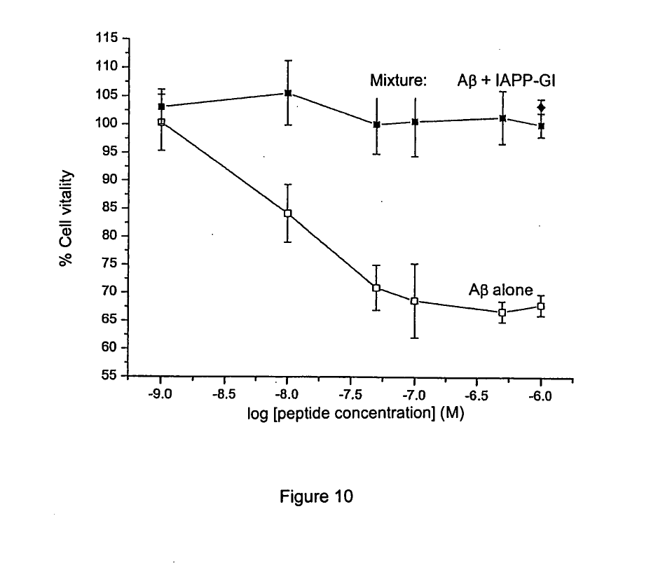

FIG. 10 shows the results of an MTT reduction test for determining the effect of the IAPP-GI analog on the cytotoxicity of A-β (PC-12 cell line). The results are average values (±SEM) from one representative assay (triplicate determination).

### EXAMPLE 1

### Characterization of the Solubility Properties of the Peptide Analogs According to the Invention, and Comparison to IAPP

The solubility properties of the peptide analogs according to the invention compared to natural IAPP were investigated using sedimentation tests, electron microscope analyses, and thioflavin-T binding tests.

a) The sedimentation test was used to investigate the solubility of the analogs compared to IAPP. The quantity of precipitated or soluble peptide was determined as a function of time. In one typical experiment, first a peptide solution at a given concentration (1, 10, or 100 μM) in 10 mM sodium phosphate buffer, pH 7.4 was prepared. Aliquots of this solution were centrifuged at specified times (20200 g, 20 min), and precipitated protein in the pellet and supernatant was quantified using the bicinchonic acid (BCA) protein determination assay. FIG. 1 illustrates several results from this test. It is seen that the aggregation of IAPP at a concentration of 10 μM began immediately after the solution was prepared. After 20 h IAPP was completely precipitated; in contrast, the N-methylated IAPP-LA [sic; IAPP-AL], IAPP-GI, and IAPP-AF [sic; IAPP-FA] analogs remained soluble over 14 days, even at a concentration of 100 μM. IAPP completely precipitated after only 2 h under the latter-referenced conditions (100 μM).

b) Amyloid fibriles from various protein species bind to the dye ThT and result in an increase in the maximum fluorescence emission of the protein. The ThT binding is therefore a specific test widely used for quantifying amyloid fibriles. This test was used to determine the amyloid formation potential of the analogs compared to IAPP. For this test the analogs and IAPP were incubated at a concentration of 62.5 μm [sic; μM] (2% HFIP, 10 mM tris, pH 7.4). 40-mL aliquots from these incubations were combined with 160 mL of a 5 μM ThT solution (in 0.1 M glycine-NaOH buffer, pH 8.5), mixed, and after excitation at 450 nm the emission from the solution was determined at 485 nm.

It was shown that IAPP forms fibriles at a concentration of 625 nM. In contrast, the analogs did not form fibriles, even at a concentration of 62.5 μM (FIG. 2).

c) For EM analyses, 5-μM solutions of the analogs versus IAPP (in 10 mM sodium phosphate buffer, pH 7.4, containing 1% HFIP) were incubated for approximately 20 h at RT. 10 μL of the solutions was then applied to EM plates, and after dyeing with 1% uranyl acetate as described in Kayed, J. Mol. Biol. (1999) 287, 781-796 was analyzed for the presence of fibriles by means of EM. It was found that, in contrast to the IAPP solution, which was composed primarily of IAPP amyloid fibriles, the solutions of the IAPP analogs contained no fibriles (FIG. 3).

Together with the ThT binding assays and the sedimentation assay results, these data show that the analogs are not amyloidogenic, and are at least 100 times more soluble than IAPP.

### EXAMPLE 2

### Investigation of the Cytotoxicity of the Analogs in Comparison to IAPP

IAPP amyloid aggregates are cytotoxic for pancreatic β-cells and for many other cells. It is assumed that the cytotoxic effect of IAPP and other amyloid polypeptides is associated with their aggregation to the amyloid. It follows that since the analogs are not amyloidogenic, they should also not be cytotoxic. To test this hypothesis the MTT cytotoxicity test, which is based on the reduction of the dye 3-[4,5-dimethylthiazole-2-yl]-2,5-diphenyltetrazolium bromide (MTT) by “healthy” cells having an intact redox potential, was used (Shearman et al., J. Neurochem. (1995) 65, 218-227; Shearman et al., Proc. Natl. Acad. Sci. USA (1994) 91, 1470-74). The latter is an early indicator of cell viability or damage. It has been shown that the cytotoxic effect of amyloid aggregates, including IAPP, may be quantified by this test. The cell toxicity of IAPP and of the analogs was investigated using the RIN5fm pancreatic cell line as described by Kapurniotu et al., J. Mol. Biol. (2002) 315, 339-350) (FIG. 4). In one typical MTT test, the peptides were prepared for incubation (5 μM in 10 mM sodium phosphate buffer with or without 1% HFIP, pH 7.4), and after 20 h at RT were applied to the cells (which had been seeded 20 h beforehand at a cell density of 5×10⁵ cells/mL). After incubation with the cells for approximately 20 h, MTT was added thereto, and after acting for 2 h the MIT reduction potential of the cells was spectrophotometrically determined. The results are reported as % cell vitality, which corresponds to the percent reduction of MIT by the control cells (100% reduction of MTT corresponds to 100% vitality), and correspond to average values (±SEM) from at least two independent tests.

As shown in FIG. 4, in contrast to IAPP the IAPP-AL, IAPP-GI, and IAPP-FA analogs showed no cytotoxicity.

### EXAMPLE 3

### Effect of the Analogs as Inhibitors or Modulators of the Amyloid Formation Potential of IAPP

The effect of the analogs on the amyloid formation potential of IAPP was investigated by electron microscopy (EM) and the ThT binding test.

In one typical ThT binding test assay, incubations were prepared which contained 6.25 μM IAPP in assay buffer (10 mM tris, pH 7.4 and 2% HFIP) alone or in a 1:1 mixture with one of the analogs. Aliquots of the solutions as described above were combined with ThT at specified times and were investigated for their fluorescence emission. As shown in FIG. 5, the analogs have a strong inhibitory effect on the amyloid formation potential of IAPP.

In one typical aggregation test tracked by EM, IAPP (5 μM) was incubated alone or in the presence of one of the analogs, in a 1:1 ratio (in 10 mM sodium phosphate buffer, pH 7.4, containing 1% HFIP). After 20 h, 10 μL of the solution was applied to an EM plate, and after dyeing with 1% uranyl acetate as described in Kayed, J. Mol. Biol. (1999) 287, 781-796 was analyzed for the presence of fibriles by means of EM. It was found that, in contrast to the IAPP solution, which was composed primarily of IAPP amyloid fibriles, fibrile formation was completely suppressed in the IAPP analog mixtures (FIG. 6).

### EXAMPLE 4

### Effect of the Analogs as Inhibitors of IAPP Cell Toxicity

The effect of the IAPP-GI, IAPP-AL, and IAPP-FA analogs on the β-cell toxicity of IAPP amyloid aggregates was investigated using two different tests. First the MTT cell viability test was used (see above). The test showed that all analogs in an IAPP/peptide analog mixing ratio of 1:1 significantly inhibited the cell toxicity of IAPP (FIG. 7A). This demonstrated that IAPP-GI is the most potent inhibitor of IAPP cytotoxicity.

In the second test, the effect of IAPP-GI on apoptotic β-cell death caused by IAPP amyloid was investigated (it is known that IAPP-mediated cell death is caused primarily by apoptosis). To this end, solutions of IAPP alone and in the presence of IAPP-GI (1:1) (5 μM in 10 mM sodium phosphate buffer, pH 7.4) were prepared, and were incubated with the plated cells at a final concentration of 500 nM for 20 h. The apoptosis was then quantified by use of a commercial apoptosis ELISA (cell death detection kit from Roche). The cytosolic nucleosomes, which are an early and specific indicator of apoptotic cell death, were quantified by use of this ELISA assay. This test also showed (FIG. 7B) that IAPP-GI is able to protect pancreatic β-cells from IAPP-induced apoptotic cell death.

### EXAMPLE 5

### Effect of the Analogs as IAPP Receptor Agonists

To test the effect of the analogs as (human) IAPP receptor agonists, first their receptor binding affinity to cells which express this receptor was tested. One example of such cells is the MCF-7 cell line (Zimmermann, et al., J. Endocrinology (1997), 155, 423-31), which originates from human breast cancer cells. Specific IAPP receptors are expressed in the MCF-7 cell line, for which reason this cell line is widely used for testing IAPP agonists/antagonists.

The receptor binding test was carried out in a manner analogous to that described by Zimmermann, et al., J. Endocrinology (1997) 155, 423-31. The binding of the rat IAPP (rIAPP) sequence labeled with ¹²⁵I, which is the strongest receptor ligand known to date, in the presence of IAPP or the IAPP-GI, IAPP-AL, or IAPP-FA peptide analogs was quantitatively determined. Mixtures of the radioactively labeled rIAPP ligands (˜80 μM with the corresponding peptides in various end concentrations (FIG. 8A) were incubated with MCF-7 cells for 1 h at RT. The cells were then washed with test buffer (the test buffer consisted of a 1:1 mixture of Dulbeccos MOD Eagle and F12 nutrient mixture (HAM) containing 0.1% BSA), combined first with NaOH (0.5 M) and then with the scintillation liquid, and the membrane-bound radioactivity was determined using a scintillation counter. As shown in FIG. 8A, all three of the analogs tested here were able to bind the IAPP receptor. IAPP-AL was shown to be the agonist having the highest receptor affinity, whereas IAPP-GI was the weakest (FIG. 8A).

To test the agonistic potential of the analogs, their adenylate cyclase activation potential (AC activation) was then tested. It has been shown that several of the receptor-mediated biological effects of IAPP are imparted by adenylate cyclase activation. The AC activation was determined by quantifying the cAMP produced after treating the cells (15 min, 37° C.) with various concentrations of IAPP or the analogs, using the cAMP Biotrak ELISA (Amersham). The test was performed on MCF-7 cells in a manner analogous to that described by Zimmermann, et al., J. Endocrinology (1997) (loc. cit.). As shown in FIG. 8B, all the analogs tested (IAPP-GI, (APP-AL, IAPP-FA) were full IAPP agonists. IAPP-AL was shown to be the most potent agonist, and is a better AC activation ligand than IAPP. IAPP-AF [sic; IAPP-FA] has the same AC activation potential as-IAPP, and IAPP-GI is a weaker agonist than IAPP.

The results of the AC activation test thus agree with the results of the receptor binding test.

### EXAMPLE 6

### Comparison of the Stability in Solution (Concentration of 1 mg/mL at pH 7.4) and Preservation of its Bioactivity (Hormonal Activity) Compared to IAPP; Potential Use as a Replacement for IAPP in Treatment

To [determine] the potential applicability of the analogs as an IAPP or pramlintide replacement in the treatment of diabetes and/or other diseases, the stability of the solutions of the analogs and thus their hormonal activity in comparison to readily aggregating IAPP was investigated. In one typical assay, aqueous solutions of IAPP, IAPP-AL, IAPP-GI, or 1:1 mixtures of the analogs with IAPP (250 μM or 1 mg/mL) in 10 μm sodium phosphate, pH 7.4, were used at RT and incubated for 4 days. The AC activation potential of these solutions was determined at various times, e.g., at times 0, 48 h, and 96 h, by means of the AC activation assay described above (end concentration in the cells was 1 μM, corresponding to maximum activation) (FIG. 9).

These experiments show that when it is stored under the above conditions, IAPP may lose more than half of its original hormonal activity (presumably because of its aggregation). In contrast, the hormonal activity of the analogs and of the IAPP/analog mixtures (1:1) was fully maintained. The selected concentration of 1 mg/mL corresponds to the concentration of a formulation of Symlin (pramlintide acetate) from Amylin Pharmaceuticals, which is used for the treatment of diabetes in clinical studies.

### EXAMPLE 7

### Inhibitory Effect of the Analogs on the Cytotoxicity of the Alzheimer Peptide β-Amyloid Peptide (A-β): Potential Use for the Treatment of Alzheimer's Disease (AD)

The effect of IAPP-GI (1:1 mixture) on the cytotoxicity of A-β was investigated by use of the MTT test. For this purpose, A-β (100 μM) alone or together with IAPP-GI was incubated at RT for 4 days in 10 mM tris buffer, pH 7.4, containing 150 mM NaCl and 2.2% HFIP. After dilution, the solutions were combined with PC-12 and HTB-14 cells. Both cell lines are frequently used for investigating the inhibitory effect of potential A-β cytotoxicity inhibitors. For both cell lines it was shown that IAPP-GI is actually able to greatly reduce the cytotoxicity of the A-β peptide (FIG. 10). The interaction of A-β with IAPP-GI was corroborated by other binding tests. Thus, IAPP-GI and other N-methylated IAPP analogs are particularly suited for the treatment of Alzheimer's disease (AD).

### EXAMPLE 8

Peptide analogs of the present invention

| SEQ | | | |
|---|---|---|---|
| ID No. | Abbreviation | Primary structure | Name |
| 1 | IAPP-GI | KCNTATCATQRLANFLVHSSNNF(N-Me)GA(N-Me)ILSSTNVGSNTY | [(N-Me)G²⁴, (N-Me)I²⁶]-IAPP |
| 2 | IAPP-AL | KCNTATCATQRLANFLVHSSNNFG(N-Me)AI(N-Me)LSSTNVGSNTY | [(N-Me)A²⁵, (N-Me)L²⁷]-IAPP |
| 3 | IAPP-FA | KCNTATCATQRLANFLVHSSNN(N-Me)FG(N-Me)AILSSTNVGSNTY | [(N-Me)F²³, (N-Me)A²⁵]-IAPP |
| 4 | IAPP-IL | KCNTATCATQRLANFLVHSSNNFGA(N-Me)I(N-Me)LSSTNVGSNTY | [(N-Me)I²⁶, (N-Me)L²⁷]-IAPP |
| 5 | IAPP-AIL | KCNTATCATQRLANFLVHSSNNFG(N-Me)A(N-Me)I(N-Me)LSSTNVGSNTY | [(N-Me)A²⁵, (N-Me)I²⁶, |
| | | | (N-Me)L²⁷]-IAPP |
| 6 | IAPP-GA | KCNTATCATQRLANFLVHSSNNF(N-Me)G(N-Me)AILSSTNVGSNTY | [(N-Me)G²⁴, (N-Me)A²⁵]-IAPP |
| 7 | IAPP-AI | KCNTATCATQRLANFLVHSSNNFG(N-Me)A(N-Me)ILSSTNVGSNTY | [(N-Me)A²⁵, (N-Me)I²⁶]-IAPP |
| 8 | IAPP-NG | KCNTATCATQRLANFLVHSSN(N-Me)NF(N-Me)GAILSSTNVGSNTY | [(N-Me)N²², (N-Me)G²⁴]-IAPP |
| 9 | IAPP-FG | KCNTATCATQRLANFLVHSSNN(N-Me)F(N-Me)GAILSSTNVGSNTY | [(N-Me)F²³, (N-Me)G²⁴]-IAPP |
| 10 | IAPP-GAIL | KCNTATCATQRLANFLVHSSNNF(N-Me)G(N-Me)A(N-Me)I(N-Me)ILSST | [(N-Me)G²⁴, (N-Me)A²⁵, |
| | | NVGSNTY | (N-Me)I²⁶, (N-Me)L²⁷]-IAPP |
| 11 | IAPP-F | KCNTATCATQRLANFLVHSSNN(N-Me)FGAILSSTNVGSNTY | [(N-Me)F²³]-IAPP |
| 12 | IAPP-G | KCNTATCATQRLANFLVHSSNNF(N-Me)GAILSSTNVGSNTY | [(N-Me)G²⁴]-IAPP |
| 13 | IAPP-A | KCNTATCATQRLANFLVHSSNNFG(N-Me)AILSSTNVGSNTY | [(N-Me)A²⁵]-IAPP |
| 14 | IAPP-I | KCNTATCATQRLANFLVHSSNNFGA(N-Me)ILSSTNVGSNTY | [(N-Me)I²⁶]-IAPP |
| 15 | IAPP-L | KCNTATCATQRLANFLVHSSNNFGAI(N-Me)LSSTNVGSNTY | [(N-Me)L²⁷]-IAPP |
| 16 | IAPP-SS | KCNTATCATQRLANFLVHSSNNFGAIL(N-Me)SSTNVGSNTY | [(N-Me)S²⁸, (N-Me)S²⁹]-IAPP |
| 17 | IAPP-ASS | KCNTATCATQRLANFLVHSSNNFG(N-Me)AIL(N-Me)S(N-Me)STNVGSNTY | [(N-Me)A²⁵, (N-Me)S²⁸, |
| | | | (N-Me)S²⁹]-IAPP |
| 18 | IAPP- | KCNTATCATQRLANFLVHSSNNFGAILSSTNVGSNTY | Natural human IAPP |

The peptide analogs according to the invention were prepared by simple chemical synthesis, in high purity and good yields according to conventional methods for solid-phase peptide synthesis on RINK amide-MBHA resin, using the Fmoc/tBu strategy (Kazantzis et al., Eur. J. Biochem. (2002) 269, 780-791). In one typical synthesis, Na-Fmoc-protected amino acids (side chain protection as follows: Lys (Boc), Cys (Tet), Arg (Pmc), Asn (Tet), His (Tet), Ser (tBu), Tyr (tBu), Thr (tBu)). N-methylated Fmoc amino acids were also used in this form (e.g., Fmoc-(N-Me) Ile; Fmoc-(N-Me) Gly, etc.). The linkages of the Fmoc amino acids (AA) were carried out using TBTU and DIEA (4× molar excess of AA, 4× excess of TBTU; 6× excess of DIEA relative to the molar quantity of C-terminal AA) in DMF and/or NMP. For the linkages of the N-methylated AA or the linkages to N-methylated AA, greater excesses, double to x-times the number of linkages, mixtures of solvents, and longer linkage times were used. Cleavage of the Fmoc group was carried out using 25% piperidine in DMF. Cleavage of the peptides from the resin was carried out using a mixture of TFA/water/thioanisol/ethanedithiol/phenol (83:4, 5:4, 5:2:6 (v/v/v/v/w)) (Kazantzis et al., Eur. J. Biochem. (2002) 269, 780-791). The resin was removed from the peptide solution by filtration, and the crude product was obtained after evaporation of the solvent, dissolving in 10% HAc, extraction with ether, and lyophilization of the aqueous phase in reduced form. Closing of the Cys²/Cys⁷ disulfide bridge was carried out in a 0.1 M NH₄HCO₃ solution (1 mg/mL peptide concentration) containing 3M GdnHCl, and required 2-4 hours. After oxidation, the crude product was again subjected to ultrahigh purification by reverse phase chromatography on a C¹⁸ column, using ACN-containing gradients (Kazantzis et al., loc. cit.).

1-43. (canceled)

44. A peptide analog selected from the group consisting essentially of SEQ. ID. NO. 1, SEQ. ID. NO. 2, SEQ. ID. NO 3, SEQ. ID. NO. 4, SEQ. ID. NO. 5, SEQ. ID. NO. 6, SEQ. ID. NO. 7, SEQ. ID NO. 8, SEQ. ID NO. 9, SEQ. ID NO. 10, SEQ. ID. 11, SEQ. ID NO. 12, SEQ. ID NO. 13, SEQ. ID NO. 14, SEQ. ID. NO. 15, SEQ. ID NO. 16, SEQ. ID. NO. 17, and functional portions thereof.

45. The peptide analog according to claim 44 wherein the peptide analog or a portion thereof binds to a natural islet amyloid polypeptide (IAPP) receptor.

46. The peptide analog according to claim 44 wherein the peptide analog or a portion thereof is an antagonist to a natural islet amyloid polypeptide (IAPP) receptor.

47. A probe comprising the peptide analog according to claim 44 and a label operably bound to the peptide analog or a portion thereof.

48. The probe according to claim 47 wherein the label is one of an acetyl group, a radioactive marker, an enzyme marker, a fluorescent marker, a luminescent marker, and a spin label.

49. The probe according to claim 47 immobilized on a substrate.

50. A pharmaceutical agent comprising at least one of the peptide analogs according to claim 44.

51. The pharmaceutical agent according to claim 50 further comprising at least one of insulin, an antibody, IAPP, and pramlintide.

52. The pharmaceutical agent according to claim 51 further comprising a pharmaceutical acceptable carrier.

53. A treatment for diabetes comprising administering an effective amount of the pharmaceutical agent according to claim 52.

54. A peptide comprising:
SEQ. ID. NO 18;
a substitution at position 1 is one of lysine and ornithine;
a substitution at position 2 is one of cysteine, aspartic acid, and 2,3-diaminopropionic acid;
a substitution at position 7 is one of cysteine, aspartic acid, and 2,3-diaminopropionic acid; and
at least one amino bond is N-methylated.

55. The peptide according to claim 54 wherein the substitution at position 2 is cysteine and the substitution at position 7 is cysteine.

56. The peptide according to claim 54 wherein the substitution at position 2 is aspartic acid and the substitution at position 7 is 2,3-diaminopropionic acid.

57. The peptide according to claim 54 wherein the substitution at position 2 is 2,3-diaminopropionic acid and the substitution at position 7 is aspartic acid.

58. The peptide according to claim 54 wherein the at least one amino bond is at least one amide bond of an α-amino group selected from the group consisting essentially of at position 22, at position 23, at position 24, at position 25, at position 26, at position 27, at position 28, and at position 29.

59. The peptide according to claim 54 wherein the peptide or a portion thereof binds to a natural islet amyloid polypeptide (IAPP) receptor.

60. The peptide according to claim 54 wherein the peptide or a portion thereof is an antagonist to a natural islet amyloid polypeptide (IAPP) receptor.

61. A probe comprising at least a portion of the peptide according to claim 54 and a label operably bound to the at least a portion of the peptide.

62. The probe according to claim 61 wherein the label is one of an acetyl group, a radioactive marker, an enzyme marker, a fluorescent marker, a luminescent marker, and a spin label.

63. The probe according to claim 62 immobilized on a substrate.

64. A pharmaceutical agent comprising the peptide according to claim 54.

65. The pharmaceutical agent according to claim 64 further comprising at least one of insulin, an antibody, IAPP, and pramlintide.

66. The pharmaceutical agent according to claim 65 further comprising a pharmaceutical acceptable carrier.

67. A treatment for diabetes comprising administering an effective amount of the pharmaceutical agent according to claim 66.

68. A method of treating a disease in a subject, the method comprising:
administering to the subject an effective amount of a pharmaceutical agent comprising at least one peptide analog of islet amyloid polypeptide (IAPP); and
improving regulation of sugar metabolism in the subject.

69. The method according to claim 68 wherein the at least one peptide analog is selected from the group consisting essentially of SEQ. ID. NO. 1, SEQ. ID. NO. 2, SEQ. ID. NO 3, SEQ. ID. NO. 4, SEQ. ID. NO. 5, SEQ. ID. NO. 6, SEQ. ID. NO. 7, SEQ. ID NO. 8, SEQ. ID NO. 9, SEQ. ID NO. 10, SEQ. ID. 11, SEQ. ID NO. 12, SEQ. ID NO. 13, SEQ. ID NO. 14, SEQ. ID. NO. 15, SEQ. ID NO. 16, SEQ. ID. NO. 17, and functional portions thereof.

70. The method according to claim 68 wherein the at least one peptide analog is a peptide comprising SEQ. ID. NO 18; a substitution at position 1 is one of lysine and ornithine; a substitution at position 2 is one of cysteine, aspartic acid, and 2,3-diaminopropionic acid; a substitution at position 7 is one of cysteine, aspartic acid, and 2,3-diaminopropionic acid; and at least one amino bond is N-methylated.

71. The method according to claim 70 wherein the substitution at position 2 is cysteine and the substitution at position 7 is cysteine.

72. The method according to claim 70 wherein the substitution at position 2 is aspartic acid and the substitution at position 7 is 2,3-diaminopropionic acid.

73. The method according to claim 70 wherein the substitution at position 2 is 2,3-diaminopropionic acid and the substitution at position 7 is aspartic acid.

74. The method according to claim 70 wherein the at least one amino bond is at least one amide bond of the alpha-amino groups selected from the group consisting essentially of at position 22, at position 23, at position 24, at position 25, at position 26, at position 27, at position 28, and at position 29.

75. The method according to claim 68 wherein the pharmaceutical agent further comprises at least one of insulin, an antibody, IAPP, and pramlintide.

76. The method according to claim 68 wherein the disease is diabetes.

77. The method according to claim 68 further comprising providing an antagonist to a natural islet amyloid polypeptide (IAPP) receptor in the subject.

78. The method according to claim 68 further comprising binding a natural islet amyloid polypeptide (IAPP) receptor in the subject.

79. The method according to claim 68 further comprising reducing aggregation of amyloid fibrils in the subject.

80. The method according to claim 68 wherein the pharmaceutical agent further comprises an antibody of the peptide analog or a portion thereof.


SEQUENCE LISTING (18 sequences)

SEQ ID NO: 1 - protein, 37 aa, Homo sapiens
  MISC_FEATURE: The amide bonds of Gly at Position 24 and Ile at Position 26 are N-methylated.
XXNTATXATQRLANFLVHSSNNFGAILSSTNVGSNTY

SEQ ID NO: 2 - protein, 37 aa, Homo sapiens; identical to SEQ ID NO: 1
  MISC_FEATURE: The amide bonds of Ala at position 25, and Leu at Position 27 are N-methylated.

SEQ ID NO: 3 - protein, 37 aa, Homo sapiens; identical to SEQ ID NO: 1
  MISC_FEATURE: The amide bonds of Phe at Position 23 and Ala at Position 25 are N-methylated.

SEQ ID NO: 4 - protein, 37 aa, Homo sapiens; identical to SEQ ID NO: 1
  MISC_FEATURE: The amide bonds of Ile at Position 26 and Leu at Position 27 are N-methylated.

SEQ ID NO: 5 - protein, 37 aa, Homo sapiens; identical to SEQ ID NO: 1
  MISC_FEATURE: The amide bonds of  Ala at Position 25, Ile at Position 26 and Leu at Position 27 are N-methylated.

SEQ ID NO: 6 - protein, 37 aa, Homo sapiens; identical to SEQ ID NO: 1
  MISC_FEATURE: The amide bonds of Gly at Position 24 and Ala at Position 25 are N-methylated.

SEQ ID NO: 7 - protein, 37 aa, Homo sapiens; identical to SEQ ID NO: 1
  MISC_FEATURE: The amide bonds of Ala at Position 25 and Ile at Position 26 are N-methylated.

SEQ ID NO: 8 - protein, 37 aa, Homo sapiens; identical to SEQ ID NO: 1
  MISC_FEATURE: The amide bonds of  Asn at Position 22 and Gly at Position 24 are N-methylated.

SEQ ID NO: 9 - protein, 37 aa, Homo sapiens; identical to SEQ ID NO: 1
  MISC_FEATURE: The amide bonds of Phe at Position 23 and Gly at Position 24 are N-methylated.

SEQ ID NO: 10 - protein, 37 aa, Homo sapiens; identical to SEQ ID NO: 1
  MISC_FEATURE: The amide bonds of Gly at Position 24,  Ala at Position 25, Ile at Position 26 and Leu at Position 27 are N-methylated.

SEQ ID NO: 11 - protein, 37 aa, Homo sapiens; identical to SEQ ID NO: 1
  MISC_FEATURE: The amide bond of Phe at Position 23 is N-methylated.

SEQ ID NO: 12 - protein, 37 aa, Homo sapiens; identical to SEQ ID NO: 1
  MISC_FEATURE: The amide bond of Gly at Position 24 is N-methylated.

SEQ ID NO: 13 - protein, 37 aa, Homo sapiens; identical to SEQ ID NO: 1
  MISC_FEATURE: The amide bond of Ala at Position 25 is N-methylated.

SEQ ID NO: 14 - protein, 37 aa, Homo sapiens; identical to SEQ ID NO: 1
  MISC_FEATURE: The amide bond of Ile at Position 26 is N-methylated.

SEQ ID NO: 15 - protein, 37 aa, Homo sapiens; identical to SEQ ID NO: 1
  MISC_FEATURE: The amide bond of Leu at Position 27 is N-methylated.

SEQ ID NO: 16 - protein, 37 aa, Homo sapiens; identical to SEQ ID NO: 1
  MISC_FEATURE: The amide bonds of Ser at Position 28 and Ser at Position 29 are N-methylated.

SEQ ID NO: 17 - protein, 37 aa, Homo sapiens; identical to SEQ ID NO: 1
  MISC_FEATURE: The amide bonds of  Ala at Position 25, Ser at Position 28 and Ser at Position 29 are N-methylated.

SEQ ID NO: 18 - protein, 37 aa, Homo sapiens [sequence omitted]